(19) United States
(12) Patent Application Publication
(10) Pub. No.: US 2003/0018257 A1
(43) Pub. Date: Jan. 23, 2003

(54) Body fat thickness measurement apparatus

(76) Inventors: E-Chang Hsu, Hsinchu (TW); Chia-Wei Tu, Hsinchu (TW); You-Ren Fang, Taoyuan (TW); Jr-Shoung Sheu, Hsinchu (TW); Li-Yi Kao, Hsinchu (TW)

Correspondence Address: NEEDLE & ROSENBERG P C, 127 PEACHTREE STREET N E, ATLANTA, GA, 30303-1811, US

(21) Appl. No.: 10/144,184

(22) Filed: May 13, 2002

(30) Foreign Application Priority Data
Jul. 5, 2001 (TW) 90116442

(51) Int. Cl. A61B 8/00

(52) U.S. Cl. 600/442

(57) ABSTRACT

A body fat thickness measurement apparatus. The apparatus comprises a transducer, an output control circuit, and a signal control device. The transducer outputs ultrasound waves with a frequency above 10 MHz and receives the ultrasound reflected back by the subject. The transducer then converts the reflected ultrasound to an analog signal. The output control circuit outputs a high voltage pulse depending on a predetermined time period or a position of the transducer. The signal control device receives the analog signal and converts it to a digital signal. A processor converts the digital signal into a video signal to be displayed on a monitor.

### BACKGROUND OF THE INVENTION

1. Field of the Invention

The present invention relates to a body fat thickness measurement apparatus, especially an apparatus for measuring body fat thickness comprising the steps of: outputting ultrasound waves into the subject, receiving and processing the ultrasound reflected by the subject, and displaying an image on a monitor for examiner to measure the body fat thickness of the subject.

2. Description of the Related Arts

Recently, it is normal to include the measurement of both body weight and body fat in health examination because it is difficult to precisely judge whether the subject's optimum weight. Having too much body fat causes many health problems, as is known by the public, therefore, people need to know not only their ideal body weight but also their body fat.

Overweight people may have excess body fat removed by surgery; however, it is a necessity to measure the body fat thickness before the surgery to avoid excess or insufficient fat removal and achieve the desired result. It is also necessary to measure body fat thickness before dieting in order to evaluate the effect of diet.

The ratio and the actual amount of the body fat can be measured by dedicated apparatus. The traditional methods for measuring body fat thickness are MRI, electrical conductivity, CT scanning, calipers, and medical ultrasound devices. However, it is not economical to measure body fat thickness by such expensive devices as MRI or CT. Also, concerns regarding the radiation of CT scanning make it unsuited for daily use. Electrical conductivity causes pain for the subject, and calipers provide only imprecise measurement due to the elasticity of tested skin. Medical ultrasound devices are mainly used for imaging internal organs with lower frequency, under 10 MHz or less, and thus are not suitable for measuring body fat.

### SUMMARY OF THE INVENTION

It is therefore a primary subject of the present invention to provide a fat measurement apparatus for surgeons, clinicians, those in beauty industry, and overweight people with precise, convenient, safe and economical measurement.

The invention outputs ultrasound waves over 10 MHz into human body by a transducer and measures body fat thickness by reading the reflection from human tissues. Depending on the density of subject tissues, the intensity of the reflection changes. Also, the intensity of the reflection becomes stronger between fat and other tissues, such as muscle or bone etc. It can be measured by the intensity of the reflection to determine body fat thickness and its distribution.

For the achievement of purposes mentioned above, this invention provides a body fat thickness measurement apparatus comprising a transducer for outputting ultrasound waves over 10 MHz into a subject in response to a high-voltage pulse and converting ultrasound waves reflected from the subject into an analog signal; an output control circuit for outputting the high-voltage pulse according to the position of the transducer; a signal processing circuit, coupled with the transducer to receive the analog signal and convert the analog signal to a digital signal; and a processor, coupled with the signal processing circuit to convert the digital signal to an image signal.

The body fat thickness measurement apparatus in the present invention further comprises a monitor, coupled with the processor to display the image signal.

Moreover, the body fat thickness measurement apparatus in the present invention further comprises a movement device for moving the transducer; and a movement detection device for detecting the position of the transducer.

Furthermore, the output control circuit of the body fat thickness measurement apparatus in the present invention comprises a timing control circuit for outputting an triggering signal according to the position of the transducer detected by the detection device; and a transmitter for outputting the high-voltage pulse triggering to the arousing signal.

As well, the signal processing circuit of the body fat thickness measurement apparatus in the present invention comprises an amplifier for amplifying the analog signal and outputting a second signal; a filter for eliminating noise from the second signal and outputting a third signal; and an analog-to-digital converter for converting the third signal to a digital signal.

In another embodiment of the present invention, the body fat thickness measurement apparatus comprises an output control circuit for outputting a high-voltage pulse in a fixed time interval; an array transducer, which comprises a plurality of transducers and is used for outputting ultrasound waves over 10 MHz to a subject in response to the high-voltage pulse and converting the ultrasound waves reflected from the subject to a corresponding analog signal; a signal processing circuit, coupled with the transducer to receive the analog signal and convert the signal to a corresponding digital signal; and a processor, coupled with the signal processing circuit to convert the digital signal to an image signal.

The body fat thickness measurement apparatus in the present invention further comprises a monitor, coupled with the processor to display the image signal.

In addition, the signal processing circuit of the body fat thickness measurement apparatus in the present invention comprises an amplifier for amplifying the analog signal and outputting a second signal; a filter for eliminating noise from the second signal and outputting a third signal; and an analog-to-digital converter for converting the third signal to the digital signal.

### BRIEF DESCRIPTION OF THE DRAWINGS

The present invention will be more fully understood and further advantages will become apparent when reference is made to the following description of the invention and the accompanying drawings in which:

FIG. 1 is a block diagram showing the units of the body fat thickness measurement apparatus in Example 1 of the present invention.

FIG. 2 is a schematic diagram showing the internal structure of sensor 10 in Example 1 of the present invention.

FIG. 3 is a flow chart showing the process of processor 14 in Example 1 of the present invention.

FIG. 4 is a block diagram showing the units of the body fat thickness measurement apparatus in Example 2 of the present invention.

FIG. 5 is a block diagram showing the array transducer 20 in Example 2 of the present invention.

FIG. 6 is a flow chart showing the process of processor 24 in Example 2 of the present invention.

### DETAILED DESCRIPTION OF THE INVENTION

This invention provides a body fat thickness measurement apparatus for measuring body fat thickness of the subject comprising a transducer, an output control circuit, a signal processing circuit, a processor and a monitor. The output control circuit outputs a high-voltage pulse depending on a predetermined time period or the position of the transducer. The transducer outputs ultrasound waves with a frequency over 10 MHz to a subject in response to the high-voltage pulse and receives the ultrasound reflected by the subject. The transducer then converts the reflected ultrasound to a corresponding analog signal. The signal processing circuit receives the analog signal and converts it into a digital signal. A processor converts the digital signal into an image signal to be displayed on a monitor.

Moreover, this invention provides a body fat thickness measurement apparatus comprising an output control circuit, a plurality of transducers (an array transducer), a signal processing circuit and a processor. The output control circuit outputs a high-voltage pulse during a constant period of time. A plurality of transducers (the array transducer) outputs ultrasound waves with a over-10 MHz frequency in response to the high-voltage pulse and receives the ultrasound reflected by the subject. The transducer then converts the reflected ultrasound to an analog signal. The signal control circuit receives the analog signal and converts it to a digital signal. The processor converts the digital signal into an image signal to be displayed on a monitor.

Without intending to limit it in any manner, the present invention will be further illustrated by the following examples.

### EXAMPLE

In accordance with the present invention, there are two examples. The sensor of the body fat thickness measurement apparatus in Example I includes a singular transducer, and the internal structure of the sensor of the body fat thickness measurement apparatus in Example II includes an array transducer with a plurality of transducers. The internal structures and the operation are described as follows:

### Example I

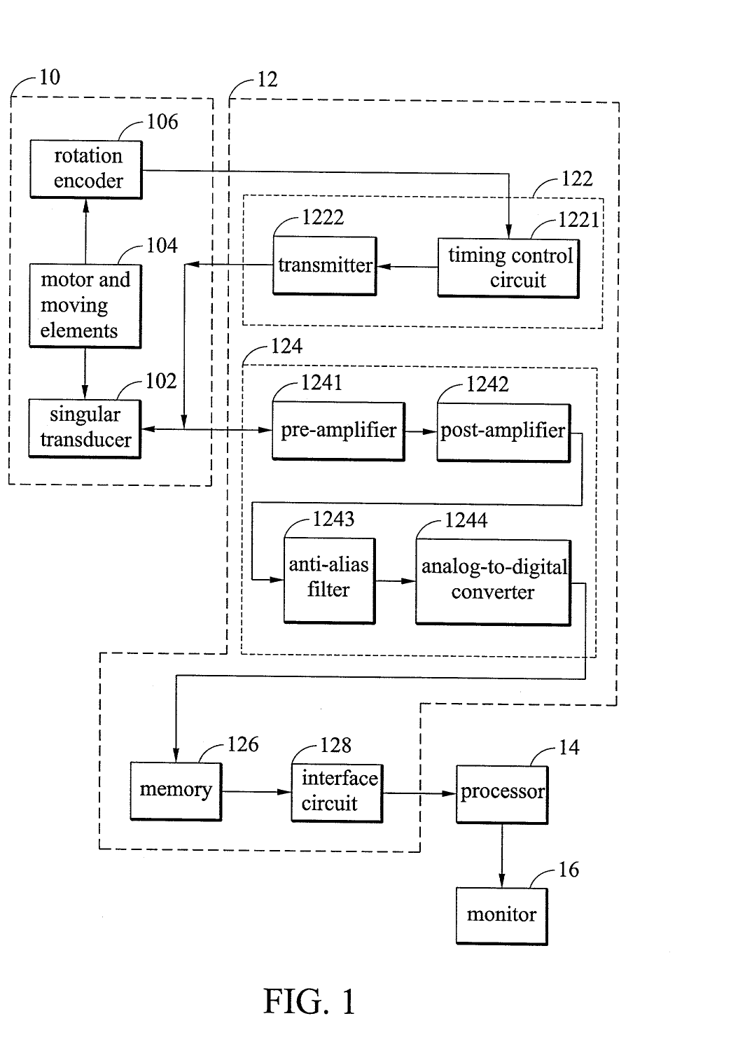

FIG. 1 is a block diagram showing the units of the body fat thickness measurement apparatus in Example I of the present invention. As shown in FIG. 1, the body fat thickness measurement apparatus in Example I of the present invention comprises a sensor 10, a system circuit 12, a processor 14, and a monitor 16.

The internal structure of the sensor 10 includes a high-frequency singular transducer 102 for outputting ultrasound waves over 10 MHz into a subject, such as human body, in response to a high-voltage pulse, and transducing the ultrasound reflected from the subject into a corresponding electrical signal, an analog signal.

In addition, the singular transducer 102 is movable by a moving device 104. The moving device 104 includes a motor and a moving element. The motor promotes the moving element, which connects to the singular transducer 102, and moves the singular transducer 102 transversely. This enhances the range of ultrasound waves sent from the singular transducer 102 into the subject. Moreover, the motor also promotes a rotation encoder 106 to rotate when the motor brings the singular transducer 102 to move transversely. According to the pulses sent back from the rotation encoder 106, the system circuit 12 receives the moving distance of the singular transducer 102.

The system circuit 12 includes an output control circuit 122, a signal processing circuit 124, a memory 126, and an interface circuit 128. The structure of these elements and the operation will be described as follows:

A timing control circuit 1221 inside the output control circuit 122 obtains the moving distance of the singular transducer 102 from the pulses sent back from the rotation encoder 106, and sends out an triggering signal to a transmitter 1222 when the singular transducer 102 moves a fixed distance. When the transmitter 1222 receives the triggering signal, it outputs a high-voltage pulse to the singular transducer 102, and the singular transducer 102 then converts the high-voltage pulse into ultrasound waves to send into the subject. In this mater, the singular transducer 102 sends out ultrasound waves every time when it moves a fixed distance.

After the transducer 102 receives the ultrasound waves reflected from the subject, the ultrasound waves are converted into a corresponding electrical signal, an analog signal, and input into a signal processing circuit 124. The signal processing circuit 124 includes a pre-amplifier 1241, a post-amplifier 1242, an anti-alias filter 1243, and an analog-to-digital converter 1244. The deeper the ultrasound waves sent out from the transducer 102 reach inside the subject, the weaker the reflected signal will be. Also, the signals reflected from different depths of the subject relate to the reflection signal received by the transducer 102. Therefore, the pre-amplifier 1241 modifies its multiples of amplification in accordance with the timing of receiving the reflection signals, and the intensity of these reflection signals will not be influenced by the depth differences of the reflection points. After that, the signal amplified by the pre-amplifier 1241 outputs to the post-amplifier 1242. The post-amplifier 1242 amplifies the signal again and the amplified signal outputs to the anti-alias filter 1243. The anti-alias filter 1243 removes high-frequency noise of the signal and outputs the signal into the analog-to-digital converter 1244. After the analog-to-digital converter 1244 converts the signal from analog to a digital signal, the signal is saved in the memory 126. When the transducer 102 finishes one round of transverse movement, the digital signals saved in the memory 126 read by the processor 14 through the interface circuit 128.

The processor 14 converts the digital signals into an image signal and outputs the image signal on the monitor 16. The process is described as follows:

Every time the transducer 102 sends out ultrasound waves, it receives reflection signals from the subject in different time points. The reflection signals are processed and displayed as a vertical line on the screen of the monitor 16. Before the vertical line is displayed, the high-frequency signals of the digital signal package are removed and only low-frequency signals are left and displayed as the vertical line. Therefore, many vertical lines are formed after the transducer 102 finishes one round of transverse movement, and the whole 2-dimensional image is formed. The non-scanning regions between the vertical lines are interpolated by the processor 14 to result in a continuous two-dimensional image. This enhances the smoothness of the image. In the image, there is a clear line between fat and other tissues of human body. Accordingly, the examiner is able to determine the body fat thickness of the subject.

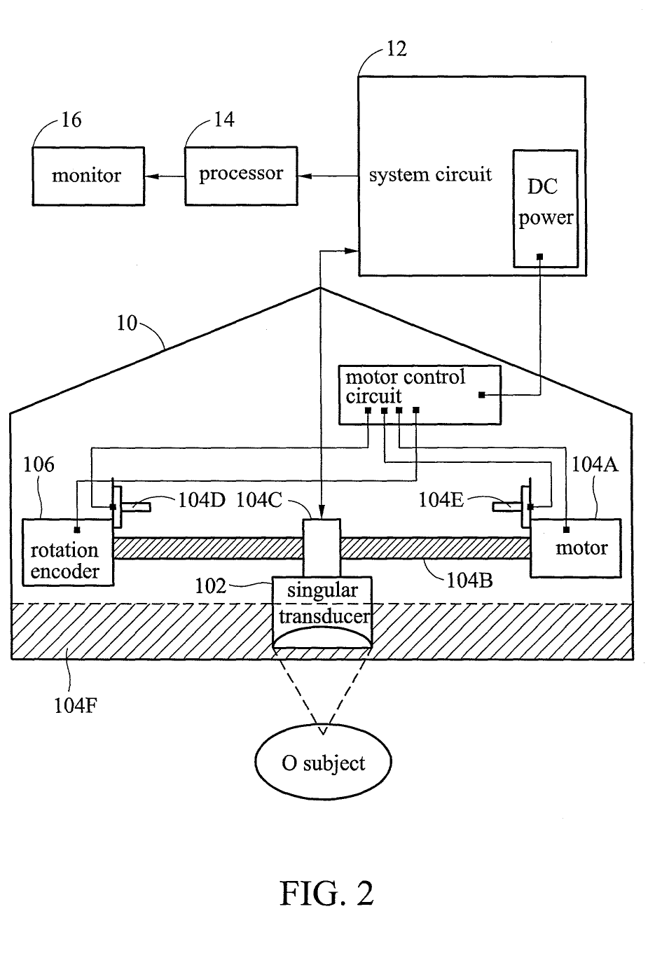

FIG. 2 is a schematic diagram showing the internal structure of the sensor 10 in Example 1 of the present invention. As shown in FIG. 2, the motor 104A promotes the screw 104B to rotate, and the moving element 104C fixed on the transducer 102 is driven. As a result, the transducer 102 moves transversely from one limit switch 104D to the other limit switch 104E. At the same time, the ultrasound waves are conducted into the subject by the coupling oil from the tank 104F.

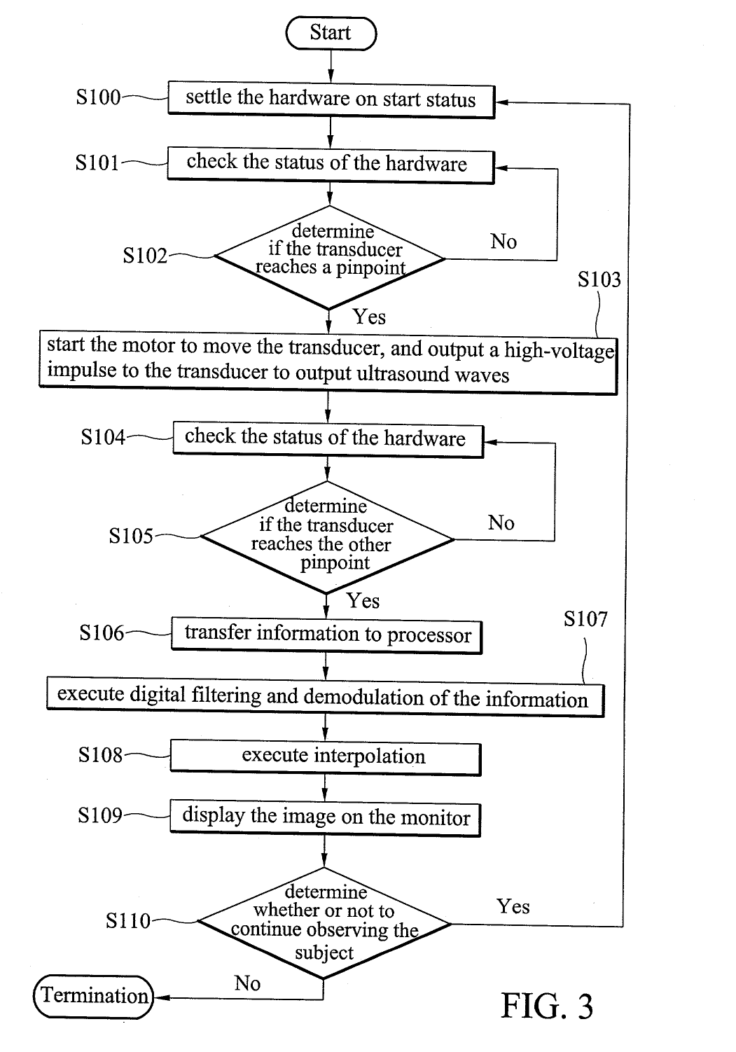

FIG. 3 is a flow chart showing the process of processor 14 in Example 1 of the present invention. First of all, the start status of the hardware is settled before operating the body fat thickness measurement apparatus in the present invention (S100). Second, the status of the hardware is checked (S101), as is the transducer 102's position, and that of the limit switch 104D (S102), to ensure that the transducer is on the initial position. If not, the process returns to S101. Next, the motor 104A is switched on to move the transducer 102, and the output control circuit 122 outputs a high-voltage pulse to the transducer 102 in order to output ultrasound waves(S103). The next step is to check that the status of the hardware is normal (S104) and to determine if the transducer 102 reaches the pinpoint, the position of the limit switch 104E (S105). If not, the process returns to step S104. When the transducer 102 reaches the limit switch 104E, the image scan is complete, and the digital signals saved in the memory 126 is transferred to the processor 14 (S106). The processor 14 executes digital filtering and demodulation of the digital signals in the beginning in order to obtain low-frequency image signals (S107). The signals are then interpolated (S108) and the processed image is displayed on the monitor 16 (S109). The apparatus awaits user instruction to run the next process (S110). If the user inputs an instruction for continuing examination, the process returns to step S100. If not, the system is terminated.

### Example II

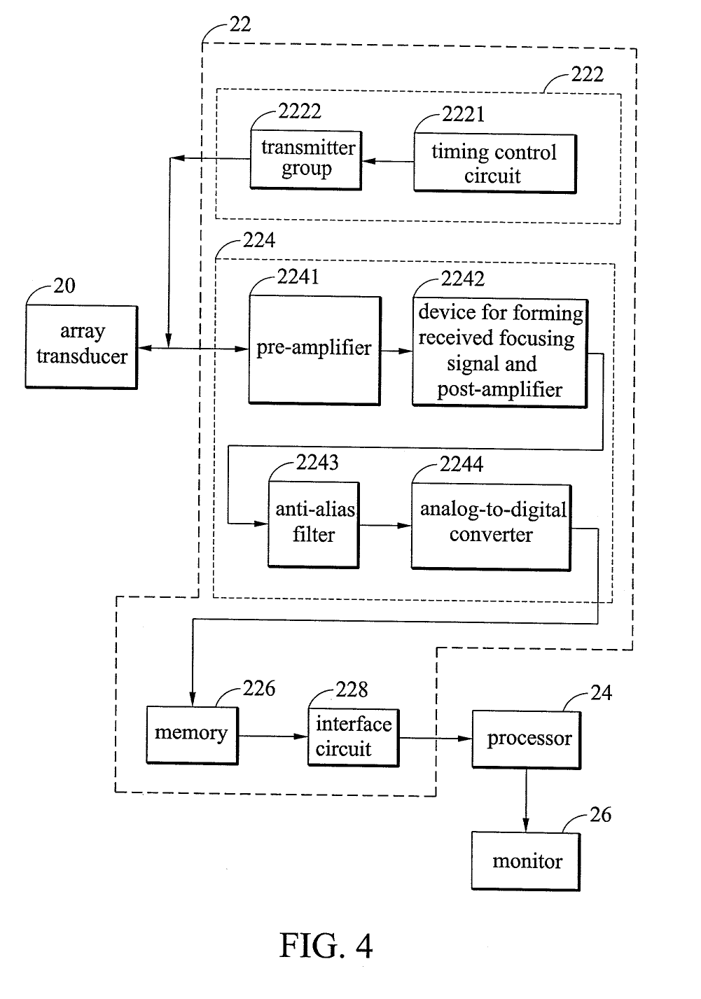

FIG. 4 is a block diagram showing the units of the body fat thickness measurement apparatus in Example 2 of the present invention. As shown in FIG. 4, the body fat thickness measurement apparatus in the present invention comprises an array transducer 20, a system circuit 22, a processor 24, and a monitor 26.

The array transducer 20 includes a plurality of transducers for outputting ultrasound waves over 10 MHz into a subject, such as human body, in response to a high-voltage pulse, and transducing the ultrasound waves reflected from the subject into a corresponding electrical signal, an analog signal.

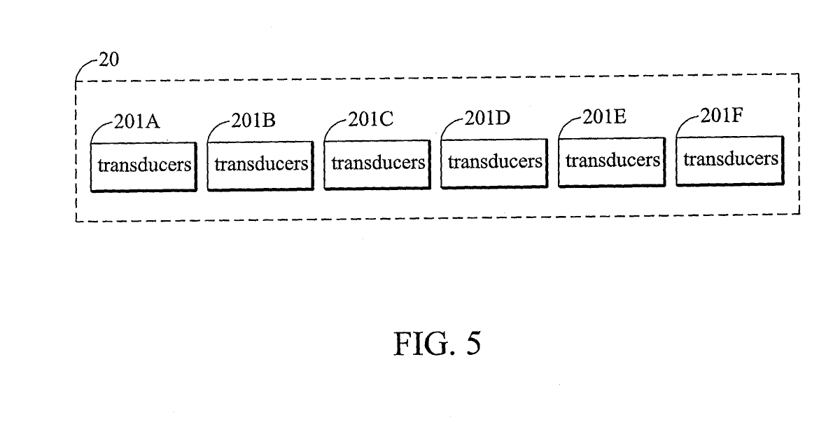

FIG. 5 is a block diagram showing the array transducer 20 in Example 2 of the present invention. As shown in FIG. 5, the array transducer 20 includes a plurality of transducers, for example, six transducers, 201A, 201B, 201C, 201D, 201E, and 201F. Each of the vertical lines of the image on the monitor 26 is composed of the signals transformed from the reflected ultrasound waves produced from a plurality of transducer, for example, three transducers. The first vertical line is composed of the group of 201A, 201B (the central signal), and 201C; the second vertical line is composed of the group of 201B, 201C (the central signal), and 201D; the third vertical line is composed of the group of 201C, 201D (the central signal), and 201E; and so on. In this order, the array transducer 20 starts from one side to the other outputting ultrasound waves group by group to the subject. In practice, the number of the transducers inside the array transducer can be hundreds, and the arrangement of the transducers can be linear or geometric.

Concerning the transmitting and receiving focusing, the timing of outputting signals for the transducers inside the array transducer 20 is different from that of inputting signals. For instance, when the transducer 201B align with the subject, the time for the transducer 201B outputting ultrasound waves must be later than that for the transducers 201A and 201C outputting ultrasound waves because the distance between the transducer 201B and the subject is shorter than that between the transducers 201A or 201C and the subject. In this matter, the ultrasound waves output by the transducers 201A, 201B and 201C are able to reach the subject at the same time (output focusing).

Similarly, when the ultrasound waves are reflected from the subject, the time for the transducer 201B receiving ultrasound waves must be earlier than that for the transducers 201A or 201C because the distance between the transducer 201B and the subject is shorter than that between the transducers 201A or 201C and the subject. Therefore, the reflected signal received by the transducer 201B is delayed, and when the signals of the transducers 201A and 201C are received, the reflected signals (input focusing) are combined and output into the signal processing circuit 224.

The system circuit 22 includes an output control circuit 222, a signal processing circuit 224, a memory 226, and an interface circuit 228. The structure of these elements and the operation will be described as follows:

A timing control circuit 2221 inside the output control circuit 222 sends out a triggering signal to the transmitter group 2222 according to the rule of transmitting and receiving focusing. When the transmitter group 2222 receives the triggering signal, it outputs a high-voltage pulse to the corresponding transducers of the array transducer 20. After receiving the high-voltage pulse, the array transducer 20 converts the high-voltage pulse into ultrasound waves to send into the subject.

After the array transducer 20 receives the ultrasound waves reflected from the subject, the reflected ultrasound waves are converted into a corresponding electrical signal, an analog signal, and input into a signal processing circuit 224. The signal processing circuit 224 includes a pre-amplifier 2241, a post-amplifier 2242, an anti-alias filter 2243, and an analog-to-digital converter 2244. The deeper the ultrasound waves sent from the transducer 20 reach inside the subject, the weaker the reflected signal will be. Also, signals reflected from different depths of the subject relate to the timing of the reflection signal received by the transducer 20. Therefore, the pre-amplifier 2241 modifies its multiples of amplification in accordance with the timing of receiving the reflection signals, and the intensity of these reflection signals will be not influenced by the depth differences of the reflection points. After that, the signal amplified by the pre-amplifier 2241 is output to the post-amplifier 2242. The post-amplifier 2242 amplifies the signal again and the amplified signal is output to the anti-alias filter 2243. The anti-alias filter 2243 removes high-frequency noise of the signal and outputs the signal into the analog-to-digital converter 2244. After the analog-to-digital converter 2244 converts the signal from analog to a digital signal, the signal is saved in the memory 226. When all transducers inside the array transducer 20 finish the output of ultrasound waves, the digital signals saved in the memory 226 are read by the processor 24 through the interface circuit 228.

The processor 24 converts the digital signals into an image signal and outputs the image signal on the monitor 26. The process is described as follows:

Every time the array transducer 20 sends out ultrasound waves, it receives reflection signals from the subject in different time points. The reflection signals are processed and displayed as vertical lines on the screen of the monitor 26. Before the vertical lines are displayed, the high-frequency signals of the digital signal package are removed and only low-frequency signals are left and displayed as the vertical lines. Therefore, many vertical lines are formed after all transducers of the array transducer 20 output ultrasound waves, and the whole 2-dimensional image is formed. The non-scanning regions between the vertical lines are interpolated by the processor 24 to result in a continuous two-dimensional image. This enhances the smoothness of the image. In the image, there is a clear line between fat and other tissues of human body. Accordingly, the examiner is able to determine the body fat thickness of the subject.

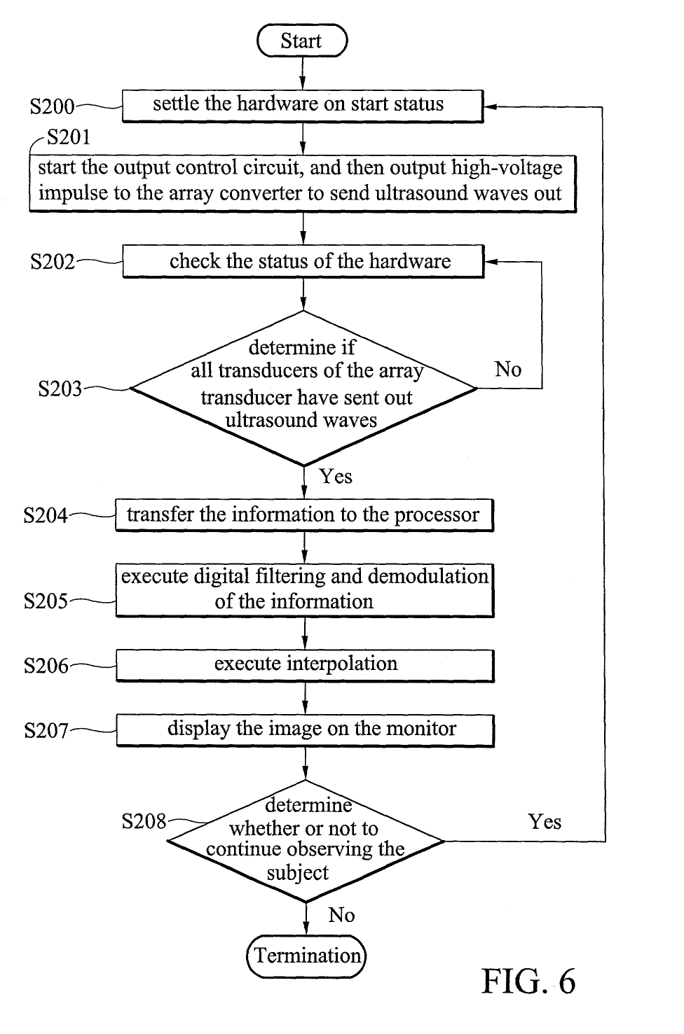

FIG. 6 is a flow chart showing the process of processor 24 in Example 2 of the present invention. First of all, the status of the hardware is settled before operating the body fat thickness measurement apparatus in the present invention (S200). Second, the output control circuit 222 is started, and it outputs high-voltage pulses to the array transducer 20 in order to send ultrasound waves out (S201). At this moment, the status of the hardware is checked (S202), as is whether all transducers of the array transducer 20 have sent ultrasound waves out (S203). If not, the process returns to S102. When all transducers of the array transducer 20 are sending ultrasound out, the whole image is complete and the digital signals saved in the memory 226 are transferred to the processor 24 (S204). The processor 24 executes digital filtering and demodulation of the received digital signals in order to obtain low-frequency image signals (S205). The signals are then interpolated (S206) and the processed image is displayed on the monitor 26 (S207). The apparatus awaits user instruction to run the next process (S208). If the user inputs an instruction for continuing examination, the process returns to step S100. If not, the system is terminated.

According to the body fat thickness measurement apparatus shown in Example I and II, the body fat thickness and its distribution on a subject are clearly displayed.

When the invention has been particularly shown and described with the reference to the preferred embodiment thereof, it will be understood by those skilled in the art that various changes in form and details may be made without departing from the spirit and scope of the invention.

What is claimed is:

1. A body fat thickness measurement apparatus, comprising:
a transducer for outputting ultrasound waves over 10 MHz into a subject in response to a high-voltage pulse and converting ultrasound waves reflected from the subject into an analog signal;
an output control circuit for outputting the high-voltage pulse according to the position of the transducer;
a signal processing circuit, coupled with the transducer to receive the analog signal and convert the analog signal to a digital signal; and
a processor, coupled with the signal processing circuit to convert the digital signal to an image signal.

2. The body fat thickness measurement apparatus as claimed in claim 1, further comprising a monitor, coupled with the processor to display the image signal.

3. The body fat thickness measurement apparatus as claimed in claim 1, further comprising:
a movement device for moving the transducer; and
a movement detection device for detecting the position of the transducer.

4. The body fat thickness measurement apparatus as claimed in claim 3, wherein the movement detection device is a rotation encoder.

5. The body fat thickness measurement apparatus as claimed in claim 3, wherein the output control circuit comprises:
a timing control circuit for outputting an triggering signal according to the position of the transducer detected by the detection device; and
a transmitter for outputting the high-voltage pulse according to the triggering signal.

6. The body fat thickness measurement apparatus as claimed in claim 5, wherein the high-voltage pulse is a short high-voltage transient signal.

7. The body fat thickness measurement apparatus as claimed in claim 1, wherein the signal processing circuit comprises:
an amplifier for amplifying the analog signal and outputting a second signal;
a filter for eliminating noise from the second signal and outputting a third signal; and
an analog-to-digital converter for converting the third signal to the digital signal.

8. The body fat thickness measurement apparatus as claimed in claim 7, wherein the amplifier modifies the multiples based on the time receiving the reflected signal.

9. The body fat thickness measurement apparatus as claimed in claim 1, wherein the processor modifies the image signal by interpolation.

10. A body fat thickness measurement apparatus, comprising:
an output control circuit for outputting a high-voltage pulse in a fixed time interval;
an array transducer, which comprises a plurality of transducers and is used for outputting ultrasound waves over 10 MHz to a subject in response to the high-voltage pulse and converting the ultrasound waves reflected from the subject to a corresponding analog signal;
a signal processing circuit, coupled with the transducer to receive the analog signal and convert the signal to a corresponding digital signal; and
a processor, coupled with the signal processing circuit to convert the digital signal to an image signal.

11. The body fat thickness measurement apparatus as claimed in claim 10, further comprising a monitor, coupled with the processor to display the image signal.

12. The body fat thickness measurement apparatus as claimed in claim 10, wherein the high-voltage pulse is a short high-voltage transient signal.

13. The body fat thickness measurement apparatus as claimed in claim 10, wherein the array transducer is a linear array transducer.

14. The body fat thickness measurement apparatus as claimed in claim 10, wherein the signal processing circuit comprises:
an amplifier for amplifying the analog signal and outputting a second signal;
a filter for eliminating noise from the second signal and outputting a third signal; and
an analog-to-digital converter for converting the third signal to the digital signal.

15. The body fat thickness measurement apparatus as claimed in claim 14, wherein the amplifier modifies the multiples based on the time of receiving the reflected signal.

16. The body fat thickness measurement apparatus as claimed in claim 10, wherein the processor modifies the image signal by interpolation.

17. The body fat thickness measurement apparatus as claimed in claim 1, wherein the subject is human body.

18. The body fat thickness measurement apparatus as claimed in claim 10, wherein the subject is human body.
